(19) United States
(12) Patent Application Publication
(10) Pub. No.: US 2026/0159828 A1
(43) Pub. Date: Jun. 11, 2026

(54) RIBONUCLEIC ACID PURIFICATION METHODS

(71) Applicant: Cytiva US LLC, Marlborough, MA (US)

(72) Inventor: Aydin Kavara, Marlborough, MA (US)

(21) Appl. No.: 19/407,642

(22) Filed: Dec. 3, 2025

Related U.S. Application Data
(60) Provisional application No. 63/729,953, filed on Dec. 9, 2024.

(51) Int. Cl. C12N 15/10

(52) U.S. Cl. CPC C12N 15/101

(57) ABSTRACT

The presently claimed and described technology provides a chromatography method for purifying ribonucleic acid (RNA) using an elution buffer with a pH of at least about 10 and comprising at least about 0.80 M of at least one salt and a neutralizing solution.

### RELATED APPLICATIONS

This application claims benefit to Provisional U.S. Application No. 63/729,953 filed Dec. 9, 2024, which is incorporated herein by reference in its entirety.

### BACKGROUND

Messenger ribonucleic acid (mRNA) has emerged as an important class of therapeutics and vaccines in recent years and purification of mRNA is a critical step that ensures the safety, efficacy, and quality of the final product. Purifying ribonucleic acid (RNA) is a challenging process due to its inherent instability and susceptibility to degradation. This intrinsic fragility necessitates stringent precautions during extraction and purification to preserve RNA integrity. The efficiency of RNA extraction and purification methods further complicates the process. Balancing high yield with the need for high purity often involves trade-offs, requiring researchers to tailor methods to their specific samples and downstream needs. These challenges highlight the critical importance of using meticulous techniques, proper equipment, and RNase-free reagents to achieve successful RNA purification.

A need exists for a high yield RNA purification method that preserves RNA integrity.

### BRIEF SUMMARY

One aspect of the disclosure is a method for purifying ribonucleic acid (RNA) comprising passing a sample comprising RNA through a chromatography device, wherein at least a portion of the RNA binds to the chromatography device; eluting the RNA from the chromatography device with an elution buffer, wherein the elution buffer has a pH of at least about 10 and comprises at least about 0.80 M of at least one salt; wherein the RNA is eluted into a neutralizing solution, wherein the neutralizing solution has a pH lower than the elution buffer; and wherein the RNA remains intact after elution.

In an aspect, the elution buffer further comprises a base selected from the group consisting of sodium hydroxide (NaOH), potassium hydroxide (KOH), calcium hydroxide (Ca(OH)₂), magnesium hydroxide (Mg(OH)₂), barium hydroxide (Ba(OH)₂), cesium hydroxide (CsOH), and combinations thereof.

In an aspect, the base is present in a concentration of between about 0.000098 M and 0.000102 M, alternatively about 0.0000982 M, alternatively about 0.0000984 M, alternatively about 0.0000986 M, alternatively about 0.0000988 M, alternatively about 0.000099 M, alternatively about 0.0000992 M, alternatively about 0.0000994 M, alternatively about 0.0000996 M, alternatively about 0.0000998 M, alternatively about 0.0001 M, alternatively about 0.0001002 M, alternatively about 0.0001004 M, alternatively about 0.0001006 M, alternatively about 0.0001008 M, alternatively about 0.000101 M, alternatively about 0.0001012 M, alternatively about 0.0001014 M, alternatively about 0.0001016 M, or alternatively about 0.0001018 M.

In an aspect, the at least one salt is selected from the group consisting of ammonium chloride, potassium chloride, sodium chloride, magnesium chloride, magnesium sulfate, magnesium nitrate, sodium citrate, ammonium sulfate, and combinations thereof.

In an aspect, the salt is present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M.

In an aspect, the pH of the elution buffer is about 10.5, alternatively about 11, alternatively about 11.5, or alternatively about 12.

In an aspect, the elution buffer further comprises at least one basic amino acid. In an aspect, the at least one basic amino acid is selected from the group consisting of arginine, histidine, and lysine.

In an aspect, the neutralizing solution comprises at least one buffering agent and/or a chelating agent. In an aspect, the buffering agent and/or chelating agent is selected from the group consisting of Tris(hydroxymethyl)aminomethane (TRIS), 2-(N-Morpholino) ethanesulfonic acid (MES), 4-(2-Hydroxyethyl) piperazine-1-ethanesulfonic acid (HEPES), 3-(N-Morpholino) propanesulfonic acid (MOPS), N-Tris(hydroxymethyl)methyl-3-aminopropanesulfonic acid (TAPSO), N-Bis(2-hydroxyethyl)-2-aminoethanesulfonic acid (BES), Piperazine-N,N′-bis(2-ethanesulfonic acid) (PIPES), N-(2-Acetamido)-2-aminoethanesulfonic acid (ACES), Ethylenediaminetetraacetic acid (EDTA), and combinations thereof.

In an aspect, the buffering agent and/or chelating agent is present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M.

In an aspect, the neutralizing solution has a pH of between about 7.5 and about 9.5; alternatively about 8.0; alternatively about 8.5; or alternatively about 9.0. In an aspect, the neutralizing solution comprises about 1.00 M Tris and has a pH of about 8.0.

In an aspect, the method further comprises washing the chromatography device with a wash solution prior to eluting the RNA. In an aspect, the wash solution comprises at least one salt is selected from the group consisting of ammonium chloride, potassium chloride, sodium chloride, magnesium chloride, magnesium sulfate, magnesium nitrate, sodium citrate, ammonium sulfate, and combinations thereof.

In an aspect, the salt is present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M.

In an aspect, the chromatography device comprises an ion exchange chromatography layer, membrane, or resin, wherein the portion of the RNA binds to the ion exchange layer, membrane, or resin.

In an aspect, the ion exchange chromatography layer, membrane, or resin is an anion exchange chromatography layer, membrane, or resin.

In an aspect, the RNA is selected from the group consisting of messenger RNA (mRNA), self-amplifying RNA (srRNA), transfer RNA (tRNA), ribosomal RNA (rRNA), small nuclear RNA (snRNA), microRNA (miRNA), small interfering RNA (siRNA), long non-coding RNA (lncRNA), Piwi-interacting RNA (piRNA), tRNA-derived small RNA (tsRNA), small rDNA-derived RNA (srRNA), circular RNA (circRNA), guide RNA (gRNA), and long guide RNA (long gRNA)

In an aspect, RNA is synthesized via in vitro transcription (IVT) or cellular transcription. In an aspect, the RNA from is synthesized from plasmid DNA (pDNA) via IVT. In an aspect, the RNA is derived from bacterial culture, cells, cell culture, cell lysate, recombinant plasmids, genetically modified plasmids, or synthetic plasmids. In an aspect, the method is carried out at temperature between about 20° C. and about 30° C.

One aspect of the method includes a kit for purifying RNA comprising a chromatography device, a elution buffer with a pH of at least about 10 and comprises at least about 0.80 M of at least one salt, a neutralizing solution with a pH lower than the elution buffer, and instructions for use.

These and other advantages, aspects, and novel features of the present disclosure, as well as details of illustrated embodiments thereof, will be more fully understood from the following description and drawings.

### BRIEF DESCRIPTION OF THE DRAWINGS

Various aspects of the present disclosure will now be described, by way of example only, with reference to the attached Figures, wherein:

FIG. 1 is a ˜6.3 kbp GFP2 plasmid for mRNA IVT.

FIGS. 2A and 2B are chromatograms depicting the elution of mRNA according to an aspect of this disclosure under basic conditions. FIG. 2A is a comparison of mRNA elution with salt and pH. FIG. 2B is a mRNA elution with 0.01 M NaOH/1 M NaCl.

FIG. 3 are electrophograms showing that mRNA purified according to an aspect of this disclosure remains intact with brief exposure to 0.01 M NaOH.

FIG. 4 is a chromatogram depicting the elution of mRNA according to an aspect of this disclosure with Arginine pH 11/1 M NaCl.

### DETAILED DESCRIPTION

### I. Introduction

RNA is inherently unstable, with its 2′-hydroxyl group on the ribose sugar making it prone to hydrolysis. This instability can lead to degradation if the elution conditions-such as temperature, pH, and ionic strength—are not carefully optimized. Additionally, elution buffers must avoid harsh conditions that might compromise RNA integrity, while still ensuring efficient recovery from binding matrices, which can sometimes require fine-tuning for different RNA types or sizes. For example, purified mRNA must maintain its integrity, stability, and proper capping to ensure efficient delivery and translation in host cells. As such, eluting intact RNA during purification is particularly challenging due to its chemical properties and the delicate nature of the purification process.

Strong binding between RNA and a purification matrix can also make elution difficult, especially for larger or structured RNAs like ribosomal RNA or certain long non-coding RNAs. Ensuring efficient elution without degrading the RNA often necessitates buffers with specific compositions which might not be optimal for all downstream applications. Conversely, incomplete elution can lead to significant loss of RNA, particularly when working with small or low-abundance samples.

Furthermore, RNase contamination remains a persistent threat during the elution step. Even small amounts of RNase introduced through reagents, equipment, or the environment can degrade the RNA during or immediately after elution. Maintaining RNase-free conditions is especially critical because the elution buffer lacks the protective inhibitors typically present earlier in the extraction process.

A need exists for a RNA purification method that preserves RNA integrity. In some aspects, the methods disclosed herein are used to purify RNA in a manner where the RNA remains intact after elution. In some embodiments, the methods include passing a sample comprising RNA through a chromatography device, wherein at least a portion of the RNA binds to the chromatography device; eluting the RNA from the chromatography device with an elution buffer, wherein the elution buffer has a pH of at least about 10 and comprises at least about 0.80 M of at least one salt; and wherein the RNA is eluted into a neutralizing solution, wherein the neutralizing solution has a pH lower than the elution buffer.

### II. Definitions

Unless otherwise defined, all technical and scientific terms used herein have the same meaning as commonly understood by one of ordinary skill in the art to which the methods described herein belong. Any reference to standard methods refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments or aspects does not imply that other embodiments or aspects are not useful and is not intended to exclude other embodiments or aspects from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. “Consisting essentially of” means including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” “one embodiment,” “an embodiment,” “certain embodiment,” or “some embodiment,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The invention is defined in the claims. However, below is a non-exhaustive listing of non-limiting exemplary aspects. Any one or more of the features of these aspects may be combined with any one or more features of another example, embodiment, or aspect described herein.

### III. Ribonucleic Acid (RNA) Purification Processes

Ribonucleic acid (RNA) is typically single-stranded, allowing it to fold into complex three-dimensional shapes. This flexibility enables RNA to perform a variety of functions beyond genetic coding, such as catalysis and molecular signaling. Its presence in both the transcription of DNA into RNA and the translation of RNA into proteins makes it central to gene expression and cellular function.

RNA exists in several forms, each serving specific roles. Messenger RNA (mRNA) carries genetic instructions from DNA to ribosomes, where it guides protein synthesis. Self-amplifying RNA (saRNA) encodes both the target protein and replication machinery, enabling its amplification within cells to produce more protein from a smaller initial dose. Transfer RNA (tRNA) and ribosomal RNA (rRNA) play key roles in the translation process by delivering amino acids and forming the structural framework of ribosomes, respectively. Non-coding RNAs, such as small nuclear RNA (snRNA), microRNA (miRNA), small interfering RNA (siRNA), long non-coding RNA (lncRNA), piwi-interacting RNA (piRNA), tRNA-derived small RNA (tsRNA), small rDNA-derived RNA (srRNA),), circular RNA (circRNA), guide RNA (gRNA), and long guide RNA (long gRNA), regulate gene expression and participate in cellular processes like RNA splicing, regulating gene expression, gene silencing, and chromatin remodeling.

Hydrophobic Interaction Chromatography (HIC) is commonly used for RNA purification; however, this method suffers from several drawbacks. It may be challenging to differentiate between RNA species with similar hydrophobic properties, leading to co-purification of impurities or incomplete separation; HIC columns may have reduced binding capacity for large RNA molecules due to steric hindrance or insufficient hydrophobic regions; and the method may exacerbate RNA degradation, especially if RNases are present or conditions are suboptimal for RNA stability.

While anion exchange chromatography is preferred, it is not employed due to the strong binding of RNA to the sorbent which can make elution challenging, even with high salt concentrations. This powerful interaction often requires harsh conditions, which risk compromising the integrity of the RNA. As a result, achieving efficient recovery while maintaining product quality can be a significant obstacle.

The inventors have developed a surprising and unexpected method that leverages an elution buffer with a pH of at least about 10 and at least about 0.80 M of at least one salt coupled with a neutralizing solution which allows for purification of intact RNA. The disclosed methods are also novel and inventive in that no desalting of the eluted intact RNA is required, only one elution buffer is needed to elute the RNA from the resin, and the inventors have shown that brief exposure to an alkaline buffer does not destroy RNA or the resin and at higher (i.e., greater than 10) pHs, the RNA is still able to be eluted from an anion exchange resin.

In an aspect, the method includes passing a sample comprising RNA through a chromatography device, wherein at least a portion of the RNA binds to the chromatography device. As used herein, “chromatography device” refers to an apparatus used in the process of chromatography. Various chromatography devices are commercially available including the Mustang™ E, Q and S chromatography membranes (Cytiva). The chromatography device may be part of a chromatography system. Various chromatography systems are commercially available including the ÄKTA avant chromatography system (Cytiva).

In some aspects, the chromatography device may be an ion exchange chromatography device. Ion exchange chromatography operates on the principle that different ions or charged molecules in a mixture will interact differently with a charged stationary phase (the resin) in the device. The stationary phase consists of ion exchange resins that can either be cationic (negatively charged to attract and bind positively charged ions) or anionic (positively charged to attract and bind negatively charged ions).

In some embodiments, the chromatography device may include an ion exchange chromatography layer, wherein at least a portion of the RNA binds to the ion exchange chromatography layer. In some embodiments, the chromatography device may include an ion exchange chromatography membrane, wherein at least a portion of the RNA binds to the ion exchange chromatography membrane. In some embodiments, the chromatography device may include an ion exchange chromatography resin, wherein at least a portion of the RNA binds to the ion exchange chromatography resin or monolith support.

Depending on the scale of purification desired, the chromatography device the bed volume may vary. The bed volume of a chromatography device refers to the total volume occupied by the stationary phase (e.g., layer, membrane, etc.). It determines the capacity for sample and buffer interactions, influencing separation efficiency and resolution. In some aspects the chromatography device may have a bed volume of about 0.5 mL, alternatively about 1 mL, alternatively about 1.5 mL, alternatively about 2 mL, alternatively about 2.5 mL, alternatively about 3 mL, alternatively about 3.5 mL, alternatively about 4 mL, alternatively about 4.5 mL, alternatively about 5 mL, alternatively about 5.5 mL, alternatively about 6 mL, alternatively about 6.5 mL, alternatively about 7 mL, alternatively about 7.5 mL, alternatively about 8 mL, alternatively about 8.5 mL, alternatively about 9 mL, alternatively about 9.5 mL, or alternatively about 10 mL.

In some aspects, the process may be used for large scale purification of RNA. In some embodiments, the RNA may be purified using a chromatography device with a bed volume of about 50 mL or greater, alternatively about 75 mL or greater, alternatively about 100 mL or greater, alternatively 125 mL or greater, or alternatively 150 mL or greater. In some aspects, the process may be scaled up or down using various commercial kits or custom protocols depending on the desired yield and purity.

In another aspect, the ion exchange chromatography layer is an anion exchange chromatography layer. In another aspect, the ion exchange chromatography membrane is an anion exchange chromatography membrane. In another aspect, the ion exchange chromatography resin is an anion exchange chromatography resin. In this aspect, the negatively charged RNA molecules bind to the positively charged functional groups on the anion exchange chromatography layer, resin, or membrane. Chromatography devices useful in the disclosed processes are described in U.S. Pub. No. 2023/0331773 and U.S. Pat. No. 7,094,347 each of which is incorporated by reference in its entirety.

In an aspect, the bound RNA may be eluted from the chromatography device with an elution buffer. The elution buffer contains components that compete with the RNA for binding sites on the chromatography layer, resin, or membrane, for example, causing the RNA to elute from the chromatography device.

In certain embodiments, the elution buffer has a pH of at least about 10, alternatively at about 10.5, alternatively about 11, alternatively about 11.5, or alternatively about 12. In certain embodiments, the elution buffer further comprises a base including, but not limited to, sodium hydroxide (NaOH), potassium hydroxide (KOH), calcium hydroxide (Ca(OH)₂), magnesium hydroxide (Mg(OH)₂), barium hydroxide (Ba(OH)₂), cesium hydroxide (CsOH), and combinations thereof. The base may be present in a concentration of between about 0.000098 M and 0.000102 M, alternatively about 0.0000982 M, alternatively about 0.0000984 M, alternatively about 0.0000986 M, alternatively about 0.0000988 M, alternatively about 0.000099 M, alternatively about 0.0000992 M, alternatively about 0.0000994 M, alternatively about 0.0000996 M, alternatively about 0.0000998 M, alternatively about 0.0001 M, alternatively about 0.0001002 M, alternatively about 0.0001004 M, alternatively about 0.0001006 M, alternatively about 0.0001008 M, alternatively about 0.000101 M, alternatively about 0.0001012 M, alternatively about 0.0001014 M, alternatively about 0.0001016 M, or alternatively about 0.0001018 M.

In certain embodiments, the elution buffer has at least about 0.80 M of at least one salt. In some embodiments, the salt is present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M. In certain embodiments, the salt may be ammonium chloride, potassium chloride, sodium chloride, magnesium chloride, magnesium sulfate, magnesium nitrate, sodium citrate, ammonium sulfate, and combinations thereof

In an embodiment, the elution buffer may also include at least one basic amino acid. A basic amino acid is an amino acid that has a side chain (R group) that is positively charged at physiological pH (around 7.4). This positive charge is due to the presence of an extra amino group (—NH₂), which tends to accept a proton (H⁺). Non-limiting examples of basic amino acids include arginine, histidine, and lysine.

In an aspect, the RNA is eluted into a neutralizing solution. A feature of the neutralizing solution is that is has a pH lower than the elution buffer. In certain embodiments, the neutralizing solution has a pH of between about 7.5 and about 9.5; alternatively about 8.0; alternatively about 8.5; or alternatively about 9.0.

In an embodiment, the neutralizing solution has at least one buffering agent and/or a chelating agent. In non-limiting aspects, the buffering agent and/or chelating agent may include Tris(hydroxymethyl)aminomethane (TRIS), 2-(N-Morpholino) ethanesulfonic acid (MES), 4-(2-Hydroxyethyl) piperazine-1-ethanesulfonic acid (HEPES), 3-(N-Morpholino) propanesulfonic acid (MOPS), N-Tris(hydroxymethyl)methyl-3-aminopropanesulfonic acid (TAPSO), N-Bis(2-hydroxyethyl)-2-aminoethanesulfonic acid (BES), Piperazine-N,N′-bis(2-ethanesulfonic acid) (PIPES), N-(2-Acetamido)-2-aminoethanesulfonic acid (ACES), Ethylenediaminetetraacetic acid (EDTA), and combinations thereof. The buffering agent and/or chelating agent may also be present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M.

In one method for purifying RNA, the neutralizing solution comprises about 1.00 M Tris and has a pH of about 8.0.

In certain embodiments, the method further comprises washing the chromatography device with a wash solution prior to eluting the RNA. In some aspects, the wash solution includes at least one salt is selected from the group consisting of ammonium chloride, potassium chloride, sodium chloride, magnesium chloride, magnesium sulfate, magnesium nitrate, sodium citrate, ammonium sulfate, and combinations thereof. The salt may be present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M.

In an aspect, the method is carried out at temperature between about 20° C. and about 30° C.; alternatively at 21° C., alternatively at 22° C., alternatively at 23° C., alternatively at 24° C., alternatively at 25° C., alternatively at 26° C., alternatively at 27° C., alternatively at 28° C., or alternatively at 29° C.

In certain aspects, the method results in a high yield or recovery of intact RNA. As used herein “% recovery” refers to the percentage of the initial amount of RNA that is successfully retrieved after a purification or processing step. A high % recovery indicates an efficient process with minimal loss of the RNA, while low % recovery suggests significant losses and potential areas for process improvement. In some aspects, % recovery may be referred to as % yield.

In some embodiments, the recovery of the purified intact RNA is between about 60% and about 100%, alternatively about 61%, alternatively about 62%, alternatively about 63%, alternatively about 64%, alternatively about 65%, alternatively about 66%, alternatively about 67%, alternatively about 68%, alternatively about 69%, alternatively about 70%, alternatively about 71%, alternatively about 72%, alternatively about 73%, alternatively about 74%, alternatively about 75%, alternatively at about 76%, alternatively at about 77%, alternatively at about 78%, alternatively at about 79%, alternatively at about 80%, alternatively at about 81%, alternatively at about 82%, alternatively at about 83%, alternatively at about 84%, alternatively at about 85%, alternatively at about 86%, alternatively at about 87%, alternatively at about 88%, alternatively at about 89%, alternatively at about 90%, alternatively at about 91%, alternatively at about 92%, alternatively at about 93%, alternatively at about 94%, alternatively at about 95%, alternatively at about 96%, alternatively at about 97%, alternatively at about 98%, or alternatively at about 99%. In some embodiments, the recovery of the purified intact RNA is greater than 100%.

In an aspect, the RNA purified is synthesized via vitro transcription (IVT) or cellular transcription. Cellular transcription is a natural process occurring within cells, driven by RNA polymerase enzymes that transcribe DNA into RNA during gene expression. In eukaryotic cells, transcription occurs in the nucleus and involves multiple steps, including the addition of a 5′ cap, splicing to remove introns, and polyadenylation to produce mature RNA. Cellular transcription ensures precise regulation of RNA synthesis in response to environmental or developmental cues, producing diverse RNA species with highly specific roles. However, this method is less suited for large-scale or modified RNA production due to its complexity and dependence on cellular machinery.

IVT is a cell-free method that uses purified enzymes, such as T7, SP6, or T3 RNA polymerases, to synthesize RNA from a DNA template containing the corresponding promoter. This highly controllable process allows for the production of custom RNA sequences with modifications, such as 5′ caps or poly(A) tails, essential for stability and functionality in applications like mRNA vaccines. IVT offers scalability and precision, enabling the rapid production of RNA for therapeutic and experimental purposes.

The RNA may also be derived from various sources depending on its intended use and production methods. These include, but are not limited to, a bacterial culture, cells, cell culture, cell lysate, recombinant plasmids, genetically modified plasmids, and synthetic plasmids. In other aspects the sample may be an environmental sample, clinical sample or food sample. The sample may be a fluid or liquid sample. In some aspects, the sample may be resuspended or reconstituted prior to passing through the chromatography device.

One aspect of the disclosure includes a kit for purifying RNA comprising a chromatography device, a elution buffer with a pH of at least about 10 and comprises at least about 0.80 M of at least one salt, a neutralizing solution with a pH lower than the elution buffer, and instructions for use.

The presently described technology and its advantages will be better understood by reference to the following examples. These examples are provided to describe specific implementations of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### EXAMPLES

### Exemplary Purification of mRNA

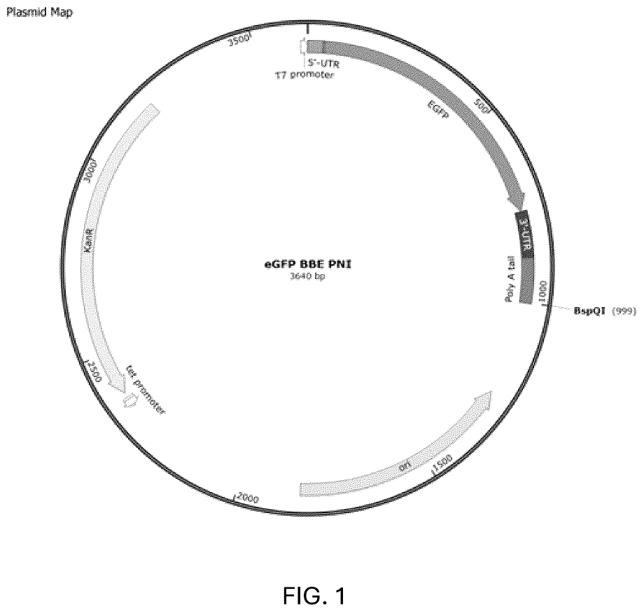

mRNA was generated using IVT reaction with standard procedures using the following plasmid: ˜6.3 kbp GFP2 plasmid for mRNA IVT Clone: E. coli DH5a, Plasmid Type: eGFP BBE PNIV201 (Precision Nanosystems Document No.: PPIS010) (FIG. 1).

Chromatography purification with 0.86 mL Mustang Q XT capsules was performed on an AKTA Avant. Materials included: Mustang Q XT 0.86 mL Cytiva Cat No.: MSTGXT25Q16; Tris(hydroxymethyl)aminomethane ACS reagent, ≥99.8% Sigma-Aldrich Cat No.: 252859; Sodium Chloride Solution 5 M Sigma-Aldrich Cat No.: S6546; Sodium Hydroxide RICCAR. Cat No.: 7460-1; and Arginine Sigma Aldrich Cat No.: A5006.

The elution yield of the XT50 modules was performed using the following method:

Representative conditions for mRNA Elution under Basic Conditions (flow rate 8.6 mL/min)
1. CIP: 5 membrane volume of 1 N NaOH
2. Equilibration 2:10 MV of 50 mM NaCl, 25 mM Tris pH 8.0, 10 mM EDTA
3. Load: mRNA (10 mL of ˜0.37 mg/mL) in Tris pH 8.0 buffer
4. Wash: 10 MV of Tris pH 9/1 M NaCl equilibration buffer
5. Elution Buffers: Arginine pH 11/1 M NaCl

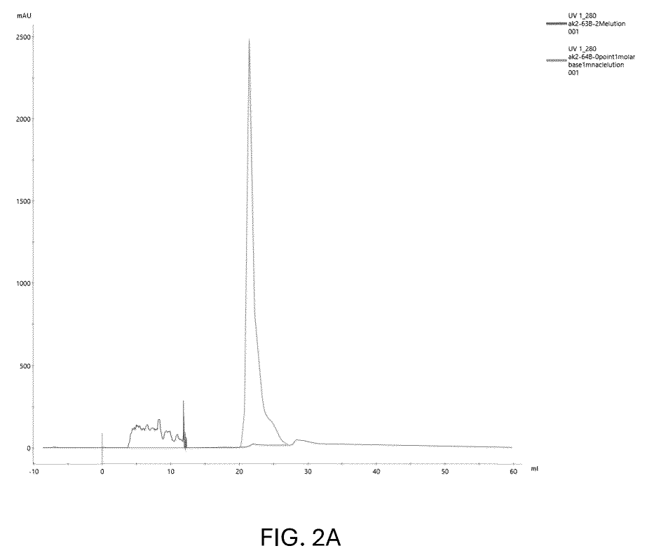

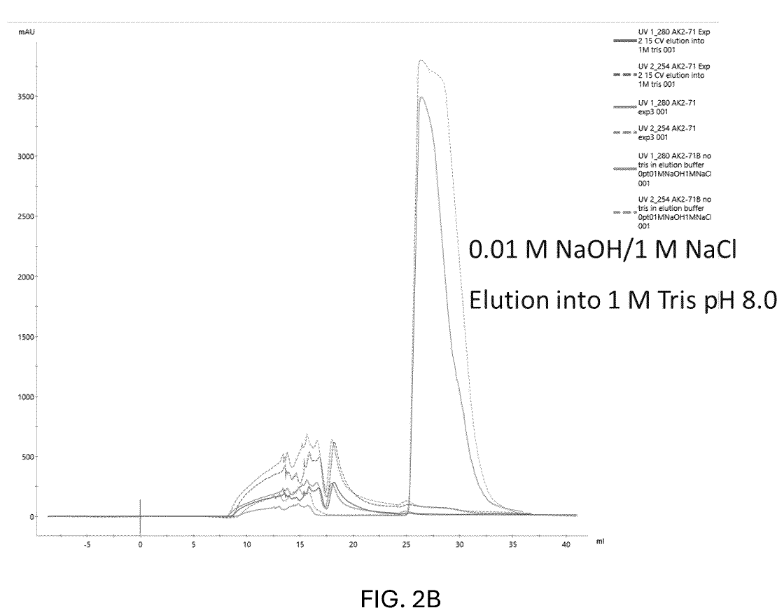

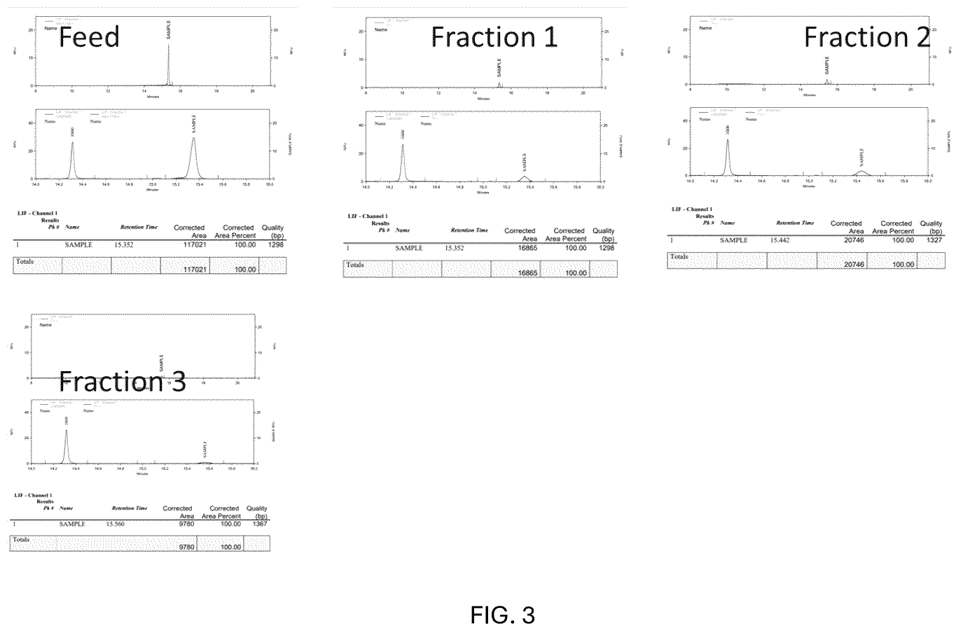

The mRNA is known not to elute from the anion exchange media under high concentration of salts. In addition, various additives, such as Arginine, Urea and Guanidine, were used to elute mRNA from Mustang Q sorbent with no success. However, when 0.01 M NaOH was used, the facile elution of mRNA into tubes containing Tris pH 8 (for neutralization of base) was observed (FIGS. 2A and 2B). As shown in FIG. 2A, mRNA does not Elute with 2 M NaCl (Blue Trace); however, elution is observed with 0.1 M NaOH/1 M NaCl. The blue and orange traces of FIG. 2B represent unsuccessful attempt at elution with pH 8.5 and pH 9. The yield of this purification procedure was measured to be more than 100% because of protein co-elution with mRNA in this experiment. Capillary electrophoresis experiments were used to verify that the mRNA stays intact under brief exposure to high pH conditions (FIG. 3).

Table 1 lists additional experiments using the methods described above with pH 9/1 M NaCl wash followed by elution under different conditions.

TABLE 1
The elution of mRNA from Mustang Q under basic conditions.

| | Concentration | | |
|---|---|---|---|
| Sample | (mg/mL) | Amount (mg) | Yield % |
| Feed | 3.78 | NA | NA |
| 0.01M NaOH/1M | 0.74 average | 5.41 | 117 |
| NaCl | | | |
| Tris pH | 0.06 | 0.72 | 38 |
| 11.3/1MNaCl* | | | |
| Arginine pH | 0.1 | 1.2 | 63 |
| 11.0/1M* NaCl | | | |

*Only 0.5 mL of starting feed was used in these experiments and pH 9/1M NaCl was employed prior to pH 11 elution.

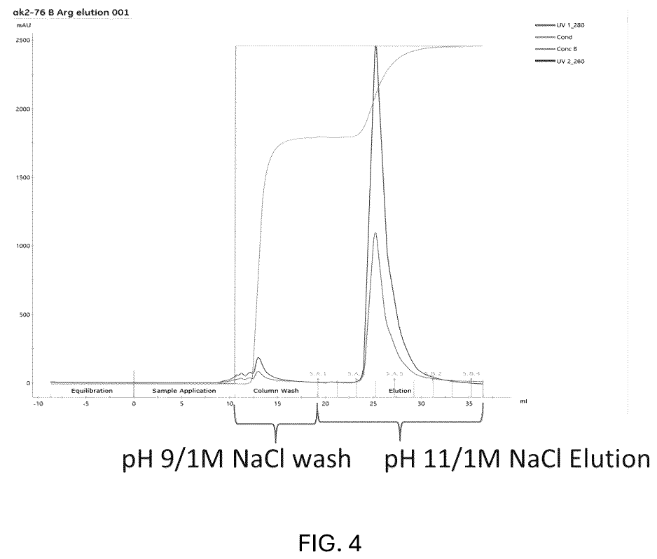

The optimal conditions were established as such: pH 9/1 M NaCl wash followed by Arginine pH 11/1 M NaCl elution (FIG. 4). This method successfully purifies the mRNA and elutes it under mildly basic conditions into tubes containing 1 M Tris/pH 8. The A260/A280 absorbance ratio and capillary electrophoresis data suggest that mRNA elutes intact with Arginine pH 11/1 M NaCl and remains intact with the brief exposure to these conditions. The mRNA is eluted into 1 M Tris pH 8 to neutralize the base, thus establishing mRNA purification method with anion exchange membrane media.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for purifying ribonucleic acid (RNA) comprising:
passing a sample comprising RNA through a chromatography device, wherein at least a portion of the RNA binds to the chromatography device;
eluting the RNA from the chromatography device with an elution buffer,
wherein the elution buffer has a pH of at least about 10 and comprises at least about 0.80 M of at least one salt;
wherein the RNA is eluted into a neutralizing solution, wherein the neutralizing solution has a pH lower than the elution buffer; and
wherein the RNA remains intact after elution.

2. The method of claim 1, wherein the elution buffer further comprises a base selected from the group consisting of sodium hydroxide (NaOH), potassium hydroxide (KOH), calcium hydroxide (Ca(OH)₂), magnesium hydroxide (Mg(OH)₂), barium hydroxide (Ba(OH)₂), cesium hydroxide (CsOH), and combinations thereof.

3. The method of claim 2, wherein the base is present in a concentration of between about 0.000098 M and 0.000102 M, alternatively about 0.0000982 M, alternatively about 0.0000984 M, alternatively about 0.0000986 M, alternatively about 0.0000988 M, alternatively about 0.000099 M, alternatively about 0.0000992 M, alternatively about 0.0000994 M, alternatively about 0.0000996 M, alternatively about 0.0000998 M, alternatively about 0.0001 M, alternatively about 0.0001002 M, alternatively about 0.0001004 M, alternatively about 0.0001006 M, alternatively about 0.0001008 M, alternatively about 0.000101 M, alternatively about 0.0001012 M, alternatively about 0.0001014 M, alternatively about 0.0001016 M, or alternatively about 0.0001018 M.

4. The method of claim 1, wherein the at least one salt is selected from the group consisting of ammonium chloride, potassium chloride, sodium chloride, magnesium chloride, magnesium sulfate, magnesium nitrate, sodium citrate, ammonium sulfate, and combinations thereof.

5. The method of claim 1, wherein the salt is present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M.

6. The method of claim 1, wherein the pH of the elution buffer is about 10.5, alternatively about 11, alternatively about 11.5, or alternatively about 12.

7. The method of claim 1, wherein the elution buffer further comprises at least one basic amino acid.

8. The method of claim 7, wherein the at least one basic amino acid is selected from the group consisting of arginine, histidine, and lysine.

9. The method of claim 1, wherein the neutralizing solution comprises at least one buffering agent and/or a chelating agent.

10. The method of claim 9, wherein the buffering agent and/or chelating agent is selected from the group consisting of Tris(hydroxymethyl)aminomethane (TRIS), 2-(N-Morpholino) ethanesulfonic acid (MES), 4-(2-Hydroxyethyl) piperazine-1-ethanesulfonic acid (HEPES), 3-(N-Morpholino) propanesulfonic acid (MOPS), N-Tris(hydroxymethyl)methyl-3-aminopropanesulfonic acid (TAPSO), N-Bis(2-hydroxyethyl)-2-aminoethanesulfonic acid (BES), Piperazine-N,N′-bis(2-ethanesulfonic acid) (PIPES), N-(2-Acetamido)-2-aminoethanesulfonic acid (ACES), Ethylenediaminetetraacetic acid (EDTA), and combinations thereof.

11. The method of claim 10, wherein the buffering agent and/or chelating agent is present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M.

12. The method of claim 9, wherein the neutralizing solution has a pH of between about 7.5 and about 9.5; alternatively about 8.0;
alternatively about 8.5; or alternatively about 9.0.

13. The method of claim 12, wherein the neutralizing solution comprises about 1.00 M Tris and has a pH of about 8.0.

14. The method of claim 1, wherein the method further comprises washing the chromatography device with a wash solution prior to eluting the RNA.

15. The method of claim 14, wherein the wash solution comprises at least one salt is selected from the group consisting of ammonium chloride, potassium chloride, sodium chloride, magnesium chloride, magnesium sulfate, magnesium nitrate, sodium citrate, ammonium sulfate, and combinations thereof.

16. The method of claim 15, wherein the salt is present in a concentration of between about 0.80 M and 1.20 M, alternatively about 0.82 M, alternatively about 0.84 M, alternatively about 0.86 M, alternatively about 0.88 M, alternatively about 0.90 M, alternatively about 0.92 M, alternatively about 0.94 M, alternatively about 0.96 M, alternatively about 0.98 M, alternatively about 1.00 M, alternatively about 1.02 M, alternatively about 1.04 M, alternatively about 1.06 M, alternatively about 1.08 M, alternatively about 1.10 M, alternatively about 1.12 M, alternatively about 1.14 M, alternatively about 1.16 M, or alternatively about 1.18 M.

17. The method of claim 1, wherein the chromatography device comprises an ion exchange chromatography layer, membrane, or resin, wherein the portion of the RNA binds to the ion exchange layer, membrane, or resin.

18. The method of claim 17, wherein the ion exchange chromatography layer, membrane, or resin is an anion exchange chromatography layer, membrane, or resin.

19. The method of claim 1, wherein the RNA is selected from the group consisting of messenger RNA (mRNA), self-amplifying RNA (srRNA), transfer RNA (tRNA), ribosomal RNA (rRNA), small nuclear RNA (snRNA), microRNA (miRNA), small interfering RNA (siRNA), long non-coding RNA (lncRNA), Piwi-interacting RNA (piRNA), tRNA-derived small RNA (tsRNA), small rDNA-derived RNA (srRNA)), circular RNA (circRNA), guide RNA (gRNA), and long guide RNA (long gRNA).

20. The method of claim 1, wherein the RNA is synthesized via in vitro transcription (IVT) or cellular transcription.

21. The method of claim 20, wherein the RNA from is synthesized from plasmid DNA (pDNA) via IVT.

22. The method of claim 21, wherein the RNA is derived from bacterial culture, cells, cell culture, cell lysate, recombinant plasmids, genetically modified plasmids, or synthetic plasmids.

23. The method of claim 1, wherein the method is carried out at temperature between about 20° C. and about 30° C.

24. A kit for purifying RNA comprising a chromatography device, a elution buffer with a pH of at least about 10 and comprises at least about 0.80 M of at least one salt, a neutralizing solution with a pH lower than the elution buffer, and instructions for use.
